(19) United States
(12) Patent Application Publication
(10) Pub. No.: US 2007/0231446 A1
(43) Pub. Date: Oct. 4, 2007

(54) Method for producing oil and fat composition with reduced trans-fatty acid content and processed oil and fat product containing the oil and fat composition

(75) Inventors: Takeshi Nagasawa, Yokosuka-shi (JP); Akira Akahane, Yokosuka-shi (JP); Kaori Muroga, Yokosuka-shi (JP); Masahiro Arikai, Yokosuka-shi (JP)

Correspondence Address: BUCHANAN, INGERSOLL & ROONEY PC, POST OFFICE BOX 1404, ALEXANDRIA, VA, 22313-1404, US

(73) Assignee: The Nisshin OilliO Group, Ltd., Tokyo (JP)

(21) Appl. No.: 11/806,222

(22) Filed: May 30, 2007

Related U.S. Application Data
(63) Continuation of application No. PCT/JP05/21863, filed on Nov. 29, 2005.

(30) Foreign Application Priority Data
Nov. 30, 2004 (JP) 2004-346922

(51) Int. Cl. A23D 9/00

(52) U.S. Cl. 426/607

(57) ABSTRACT

A method for producing an oil and fat composition includes the steps of (a) mixing an oil and fat (A) wherein fatty acids with 14 or less carbon atoms are contained in an amount of 50 to 100% by mass of the entire constituent fatty acids of the triglycerides with an oil and fat (B) wherein fatty acids with 16 to 24 carbon atoms are contained in an amount of 80 to 100% by mass of the entire constituent fatty acids of the triglycerides to obtain an oil and fat mixture; (b) subjecting the oil and fat mixture to random transesterification using an enzyme having no positional specificity, and (c) obtaining an oil and fat composition free from trans-fatty acid.

### TECHNICAL FIELD OF THE INVENTION

The present invention relates to a method for producing an oil and fat composition containing no trans-fatty acid and having a reduced saturated fatty acid content, which composition is designed to manufacture processed oil and fat food products, and to a processed oil and fat food product that is obtainable using the above-mentioned oil and fat composition.

### BACKGROUND ART

Most of the processed oil and fat food products including a margarine and a shortening are prepared by hardening oils and fats in a liquid form, such as soybean oil, rapeseed oil, cottonseed oil and the like through hydrogenation to obtain a proper hardness for use within the operating temperature region. Palm oil or palm fractions with high melting points, which partially remain unmelted even at as high as 35° C. or more are considered to have little commercial importance when used for the processed oil and fat food products due to an unpleasant flavor and lack of a smooth melting texture. In contrast to this, when the above-mentioned liquid oils and fats are subjected to hydrogenation with the degree of hydrogenation and the reaction selectivity being taken into consideration, and the resultant hardened oils and fats (hereinafter, also referred to “hydrogenated (hardened) oils and fats”) are used for manufacture of processed food products, the hardened oils and fats can maintain adequate plasticity for obtaining excellent processability in the manufacture of the processed food products, and the obtained food products can smoothly melt below the body temperature to offer a pleasant melting texture and a distinctive mellow flavor when placed in the mouth.

The plasticity within a temperature region from 5 to 25° C. and the rapid melting performance at 25° C. or more are physical properties typically found in the hydrogenated (hardened) oils and fats, especially prepared from liquid oils and fats as raw materials, which properties are related to the solid fat contents for the oil and fat composition at various temperatures. Such physical properties are considered to result from the presence of fatty acid having a double bond in the “trans” configuration in the glyceride molecule, which occurs in the course of hydrogenation (for example, see Patent Document 1)

Large quantities of the fatty acid having a double bond in the trans configuration (hereinafter referred to as trans-fatty acid) do not naturally occur. Some medical investigations conducted in the European countries and the U.S.A. have demonstrated that the trans-fatty acid acts to increase the total blood cholesterol level and the level of low density lipoprotein-cholesterol (generally called “bad cholesterol”) in the blood in the same fashion as the saturated fatty acid when the animals including the human take large quantities of trans-fatty acid over a long period of time. Further, it has been clarified that the above-mentioned cholesterol-raising effects of the trans-fatty acid would cause obesity and heart diseases such as an ischemic heart disease and the like. To promote and maintain the good health of the people, some countries are advising the people to restrict the intake of trans-fatty acids, and at the same time, requiring processed food manufacturers to list trans-fatty acids as well as saturated fatty acids on food labels if their contents are above the prefixed levels.

In line with such international tendency, oils and fats substitutable for the hydrogenated (hardened) oils and fats have been developed. Most of oils and fats rich in lauric acid, for example, coconut oil and palm kernel oil or their hydrogenated (hardened) oils and fats show the profile that the solid fat contents are high within a temperature region of 5 to 15° C. and abruptly decrease at 20 to 35° C., and the solid fats completely melt at the temperature equal or higher than the body temperature. The solid fat content curves of these oils and fats are similar to those of the hydrogenated (hardened) oils and fats obtained by subjecting the liquid oils and fats to hydrogenation, although there is a difference in the melting point.

When the lauric type oils and fats are used to reduce the trans-fatty acids, fine adjustments are needed to obtain an appropriate melting point and proper solid fat contents within the operational temperature range. To achieve the adjustments, for example, higher melting point fractions of the medium-melting point oils and fats and fully hydrogenated oils and fats (hereinafter, also referred to as “extremely hardened oils and fats”) made from liquid oils and fats are blended into the composition of oil and fat. However, it is known that the oil and fat composition prepared by this method offers an absolutely awful melting texture (for example, see Patent Document 2).

Although there are some similarities in the solid fat content curves, all the physical properties of the lauric type oils and fats are not similar to those of the hydrogenated (hardened) oils and fats obtained by subjecting the liquid oils and fats to hydrogenation. To realize the physical properties analogous to those of the hydrogenated oils and fats, high content of lauric type oils and fats becomes necessary. In this case, however, there occur another problems not found in the case of the hydrogenated (hardened) oils and fats, which makes it difficult to substitute the lauric type oils and fats for the hydrogenated oils and fats. To be more specific, the lauric type oils and fats tend to form hard crystals because of the high solid fat contents within a low temperature region from 0 to 10° C., thereby decreasing the plasticity. As compared with the hydrogenated oils and fats, the lauric type oils and fats show more significant contraction when the crystallization takes place, so that the processed oil and fat products made from the lauric type oils and fats are subject to cracking and loss of shape, which may substantially impair the commercial product value. In addition, the lauric type oils and fats are subject to hydrolysis because the polarity of the constituent fatty acids is high. When the contents of the lauric oils and fats are increased in the composition, the soapy smell peculiar to those kinds of oils and fats may generate during the storage of the oil and fat composition or after manufacture of the processed oil and fat product, which often decreases the commercial importance.

Patent Document 1: Japanese Patent Unexamined Publication (JP Kokai) Sho 53-94066

Patent Document 2: JP Kokai Sho 47-13607

### DISCLOSURE OF THE INVENTION

### Problems to be Solved by the Invention

An object of the present invention is to provide a method for producing an oil and fat composition free of trans-fatty acid, which has excellent plasticity and can melt smoothly in the mouth to feel good on the tongue, and to provide a processed oil and fat product that can be obtained by using the above-mentioned oil and fat composition.

### Means of Solution of the Problems

The inventors of the present invention have paid attention to the following points: there are similarities in the solid fat content curve between the lauric type oils and fats and the hydrogenated (hardened) oils and fats; rapid melting performance within a temperature region from medium to high temperatures is considered to be ascribed to the contents of medium-chain fatty acids having 6 to 14 carbon atoms; and fatty acids are unevenly coordinated in a triglyceride molecule of vegetable oils and fats, i.e., a fatty acid composition in the sn-2 position is different from that in the sn-1 position or sn-3 position, while the lauric type oils and fats and the hydrogenated (hardened) oils and fats thereof often exhibit the coordination of lauric acid in the sn-2 position of the triglyceride molecule. In light of the above, the inventors of the present invention found that a trans-fatty acid-free oil and fat composition can be created by using as the raw materials fatty acids with particular configurations and carrying out random transesterification using an enzyme having no positional specificity.

Namely, the present invention provides a method for producing an oil and fat composition, comprising the steps of:

(a) mixing an oil and fat (A) wherein fatty acids with 14 or less carbon atoms are contained in an amount of 50 to 100% by mass of the entire constituent fatty acids of triglycerides, with an oil and fat (B) wherein fatty acids with 16 to 24 carbon atoms are contained in an amount of 80 to 100% by mass of the entire constituent fatty acids of triglycerides, to obtain an oil and fat mixture;

(b) subjecting the fat and oil mixture to a random transesterification reaction using an enzyme having no positional specificity; and

(c) obtaining an oil and fat composition free from trans-fatty acid.

The present invention also provides an oil and fat composition that can be obtained by the above-mentioned method.

Further, the present invention provides a method for producing a processed oil and fat product using the above-mentioned oil and fat composition, and a processed oil and fat product containing the above-mentioned oil and fat composition.

### Effects of the Invention

The oil and fat composition of the present invention has excellent plasticity and can smoothly melt when placed in the mouth to feel good on the tongue because the solid fat content curve is similar to those of the hydrogenated (hardened) oils and fats made from liquid oils and fats such as rapeseed oil and the like through hydrogenation. The present invention can provide an oil and fat composition containing no trans-fatty acid hazardous to the human body. By using the oil and fat composition of the present invention, there can be obtained processed oil and fat products such as a margarine, shortening, cream and the like which are advantageous in terms of the plasticity and the smooth melting texture. For example, when prepared from the oil and fat composition of the present invention, roll-in margarine shows excellent extensibility and cream is excellent in shape retention properties and water absorption properties. Further, the oil and fat composition of the present invention can be partially or entirely substituted for hydrogenated (hardened) oils and fats usually contained in the processed oil and fat products, which makes it possible to reduce the contents of trans-fatty acids and saturated fatty acids.

### BEST MODE FOR CARRYING OUT THE INVENTION

In the present invention, the raw oils and fats and/or triglycerides that can be obtained after random transesterification may be subjected to hydrogenation.

For the hydrogenation of the raw oils and fats, the above-mentioned oil and fat (A) and oil and fat (B) may individually be subjected to hydrogenation, or either the oil and fat (A) or (B) may be subjected to hydrogenation, prior to the step (a) of mixing the oil and fat (A) and the oil and fat (B) together. Alternatively, prior to the step (b) where the oil and fat mixture of (A) and (B) is subjected to random transesterification, the oil and fat mixture may be hydrogenated.

For the hydrogenation of the triglycerides obtained by the random transesterification, an oil and fat composition that can be obtained after the random esterification of the step (b) may be subjected to hydrogenation.

Preferably, the hydrogenation may be carried out to such an extent that the obtained iodine value reaches 5 or less, more preferably 1 or less. The hydrogenation may be carried out by placing a raw oil and fat into a stirrer and a tank equipped with a heating and cooling function, to which a pressure may be applied in a controllable manner, and heating the raw oil and fat to about 120° C. while stirring under reduced pressure, to remove the remaining water content, and then carrying out a hydrogenation reaction under the conditions suitable for obtaining desired physical properties. In general, when an edible oil and fat is prepared by hydrogenation, the parameters for determining the physical properties and productivity of the edible oil and fat includes the following four conditions, i.e., the concentration of catalyst, reaction temperature, hydrogen pressure, and stirring speed. Hydrogenation for the edible oils and fats is usually carried out to obtain the targeted physical properties, with the degree of hydrogenation and the reaction selectivity being taken into consideration. The production of the oil and fat composition of the present invention, however, needs complete hydrogenation and does not need to consider the reaction selectivity, so that the reaction may preferably be carried out at high concentration of catalyst, high reaction temperature, high hydrogen pressure and high stirring speed.

There are specific embodiments as follows.

In a first embodiment, the method comprises the steps of individually subjecting an oil and fat (A) and an oil and fat (B) to hydrogenation until the iodine value of 5 or less is obtained, mixing the hydrogenated (hardened) oil and fat (A) with the hydrogenated (hardened) oil and fat (B) to obtain an oil and fat mixture, subjecting the oil and fat mixture to random transesterification, and obtaining an oil and fat composition free from trans-fatty acid.

According to a second embodiment, the method comprises the steps of mixing an oil and fat (A) with an oil and fat (B) to prepare an oil and fat mixture, subjecting the oil and fat mixture to random transesterification, carrying out hydrogenation, and then obtaining an oil and fat composition free from trans-fatty acid.

In a third embodiment, the method comprises the steps of subjecting an oil and fat (B) to hydrogenation until its iodine value reaches 5 or less, mixing the hydrogenated (hardened) oil and fat (B) with an oil and fat (A) to obtain an oil and fat mixture, subjecting the oil and fat mixture to random transesterification, and obtaining an oil and fat composition free from trans-fatty acid.

In a fourth embodiment, the method comprises the steps of subjecting an oil and fat (A) to hydrogenation until its iodine value reaches 5 or less, mixing the hydrogenated (hardened) oil and fat (A) with an oil and fat (B) to obtain an oil and fat mixture, subjecting the oil and fat mixture to random transesterification, and obtaining an oil and fat composition free from trans-fatty acid.

In a fifth embodiment, the method comprises the steps of mixing an oil and fat (A) with an oil and fat (B) to obtain an oil and fat mixture, subjecting the oil and fat mixture to hydrogenation until its iodine value reaches 5 or less, thereafter subjecting the mixture to random transesterification, and obtaining an oil and fat composition free from trans-fatty acid.

In the above-mentioned first, third and fourth embodiments, hydrogenation may further be carried out after completion of the random transesterification. Of those embodiments, the first, second and third embodiments are preferable, the first and second embodiments are more preferable, and the first embodiment is most preferable. Any embodiments can produce an oil and fat composition conveniently and economically.

<Oil and Fat (A)>

An oil and fat (A) is the one wherein fatty acids having 14 or less carbon atoms, preferably 6 to 14 carbon atoms are contained in an amount of 50 to 100% by mass, preferably 50 to 80% by mass, more preferably 60 to 80% by mass, with respect to the total mass of the constituent fatty acids of the triglycerides. It is preferable to use an oil and fat (A) containing fatty acids with 12 or less carbon atoms in an amount of 45 to 100% by mass, preferably 50 to 70% by mass, with respect to the total mass of the constituent fatty acids of the triglycerides. In particular, the use of lauric type oils and fats is preferred. To be more specific, it is preferable to use an oil and fat (A) selected from the group consisting of coconut oil, palm kernel oil, fractionated products thereof, medium chain triglycerides, and mixtures thereof. The hardened (hydrogenated) oils and fats made from the above-mentioned oils and fats may be used. The medium chain triglyceride herein used is a triacylglycerol where the constituent fatty acids have 8 to 12 carbon atoms, including the ones artificially synthesized by conventional methods. Such lauric type oils and fats have high lauric acid contents, and for example, include a commercial product of refined coconut oil (available from The Nisshin Oilio Group, Ltd.), a commercial product of hardened palm kernel oil (available from The Nisshin Oilio Group, Ltd.), and a commercial product of RBD palm kernel oil (available from Mitsubishi Corporation.).

<Oil and Fat (B)>

An oil and fat (B) is chosen from vegetable oils and fats such as palm oil and palm fractions, soybean oil, rapeseed oil, cottonseed oil and the like, wherein fatty acids having 16 to 24 carbon atoms, preferably 16 to 18 carbon atoms are contained in an amount of 80 to 100% by mass, preferably 90 to 100% by mass, more preferably 95 to 100% by mass, and more preferably 97.8 to 100% by mass with respect to the total mass of the constituent fatty acids of the triglycerides. The hardened (hydrogenated) oils obtained from the above-mentioned oils and fats may be used. More specifically, a commercial product of fully hydrogenated palm oil (extremely hardened palm oil) (available from Yokozeki Oil & Fat Corp.), a commercial product of refined palm oil (available from The Nisshin Oilio Group, Ltd.) and the like can be used.

Preferably, the oil and fat (A) and the oil and fat (B) may be used at a ratio by mass of (4:6) to (8:2), more preferably (4:6) to (6:4). In the case where the oil and fat (A) and/or the oil and fat (B) is subjected to hydrogenation prior to the transesterification, it is preferable to obtain the above-mentioned (A):(B) ratio after the hydrogenation. When the ratio of the oil and fat (A) to the oil and fat (B) is within the above-mentioned range, the solid fat content curve becomes similar to those of the hardened (hydrogenated) oils and fats made from liquid oils and fats such as soybean oil and rapeseed oil, and finally, there can be obtained an oil and fat composition with a smooth melting texture.

Although lipase from Candida sp. is one of the enzymes having no positional specificity, lipase from Alcaligenes sp. is considered to form the best mode of the present invention because this kind of lipase is most suitable for industrial-scale production from the viewpoints of the activity and heat resistance of the enzyme. Preferably, the lipase may not be immobilized. In particular, the lipase in a powdery form is preferred according to the present invention. More specifically, a commercially available lipase product “Lipase QLM” (made by Meito Sangyo Co., Ltd.) can be preferably used. The use of “Lipase QLM” is advantageous because this lipase shows a high initial reaction speed, a high optimum reaction temperature and excellent heat resistance, and in addition, this enzyme can be collected after completion of the reaction to reduce the cost of catalyst.

The advantage from the use of lipase in a powdery form is in that the transesterification can be achieved by dispersing the powdery lipase in an oil and fat mixture in the presence or absence of an inactive organic solvent. Preferably 0.05 to 1 part by mass, more preferably 0.1 to 0.5 parts by mass of the powdery lipase may be used with respect to 100 parts by mass of the oil and fat mixture. In this case, the lipase particles dispersed in the inactive organic solvent may preferably be subjected to such a treatment that can make the particle diameter of the dispersed particles uniform. For example, ultrasonic waves of 20 to 150 kHz may be applied to the lipase-containing solvent for 1 to 30 minutes so that 90% or more of the dispersed lipase particles may preferably have a particle diameter of 1 to 100 μm, more preferably 20 to 50 μm in the transesterification reaction. This can prevent the formation of what is called “clumps”. The inactive organic solvent such as a hydrocarbon including hexane, heptane or the like may be used in an amount of 10 to 90 parts by mass with respect to 100 parts by weight of the raw oil and fat materials.

The random transesterification reaction may preferably be carried out under normal pressure at a temperature around the optimal temperature of the enzyme to be used over a period of 5 to 20 hours in accordance with Japanese Patent No. 2,668,187. As mentioned above, the inactive organic solvent may be used in the transesterification. In the present invention, the oil and fat (A) and the oil and fat (B) serving as the raw materials, and the resultant oil and fat composition can appropriately be deodorized and bleached by conventional methods. The triglycerides are recovered by the conventional method after completion of the reaction, and further refined if necessary, so that an oil and fat composition free from trans-fatty acid according to the present invention can be obtained.

After completion of the random transesterification reaction, hydrogenation may be carried out if necessary, thereby generating an oil and fat composition free of trans-fatty acid.

In the present invention, the trans-fatty acid content can be measured using a capillary gas chromatography equipped with a flame ionization detector (FID), the temperature being raised from 100 to 250° C. It is preferable that the oil and fat composition of the present invention have an iodine value of 5 or less, and more preferably 1 or less. The iodine value herein used can be determined using Wijs reagent under the conditions as described in “Standard Methods for the Analysis of Fats, Oils and Related Materials” (by Japan Oil Chemists' Society). The obtained oil and fat composition may have a slip melting point (in open capillary tubes) of 40 to 55° C., more preferably 42 to 50° C. The slip melting point can be determined using a glass capillary (made by Drummond Scientific Company) under the conditions as described in “Standard Methods for the Analysis of Fats, Oils and Related Materials” (by Japan Oil Chemists' Society).

In the oil and fat composition that can be obtained by the method of the present invention, lauric acid residues are widely distributed over the triglyceride molecules. This can be confirmed by the fatty acid analysis of the oil and fat composition in the sn-2 position of triglyceride through enzymolysis.

By using the oil and fat composition obtained by the method of the present invention, processed oil and fat products, for example, margarine, roll-in margarine, shortening, cream and chocolate can be produced. The trans-fatty acid contents and the saturated fatty acid contents of the processed oil and fat products can be reduced by entirely or partially replacing the hardened (hydrogenated) oils and fats conventionally used for processed oil and fat products by the oil and fat composition obtained of the method of the present invention. To be more specific, the oil and fat composition of the present invention is mixed with a liquid oil and fat, e.g., a vegetable oil in a liquid form including rapeseed oil, soybean oil and the like, preferably a liquid vegetable oil with a high oleic acid content and a low linolenic acid content (e.g., canola oil with high oleic acid content and low linolenic acid content). The resultant mixture is then mixed with water and the like to form an emulsion by stirring, thereby producing margarine, shortening and the like. In this case, it is preferable that the ratio by mass of the oil and fat composition of the present invention to the liquid oil and fat be in the range of (8:92) to (25:75), more preferably (10:90) to (18:82). When these two components are blended at the above-mentioned ratio, the resultant processed oil and fat products have reduced trans-fatty acid contents, and at the same time, the products can smoothly melt in the mouth and show excellent plasticity.

In the margarines and shortenings thus produced using the oil and fat composition obtainable by the method of the present invention, the contents of the saturated fatty acids may preferably be in the range of 8 to 60% by mass, more preferably in the range of 8 to 40% by mass, and further preferably in the range of 10 to 20% by mass, with respect to the entire constituent fatty acids.

Other features of this invention will become apparent in the course of the following description of exemplary embodiments, which are given for illustration of the invention and are not intended to be limiting thereof.

### EXAMPLES

### Example 1

Raw materials, that is, an oil and fat (A) and an oil and fat (B) as shown in Table 1 were used to produce an oil and fat composition No. 1.

TABLE 1

| | Oil and Fat (A) | Oil and Fat (B) |
|---|---|---|
| | Hydrogenated | Fully hydrogenated |
| | palm kernel oil*¹ | palm oil*² |
| Content of fatty acids | 69.5% by mass | 2.1% by mass |
| (C14 or less) | | |
| Content of fatty acids | 53.5% by mass | |
| (C12 or less) | | |
| Content of fatty acids | 30.5% by mass | 97.9% by mass |
| (C16-C24) | | |
| Trans-fatty acid content | 0% by mass | 0% by mass |
| Iodine value | 0.31 | 0.38 |

*¹Commercially available product name: Hydrogenated palm kernel oil (made by The Nisshin Oilio Group, Ltd.)
*²Commercially available product name: Fully hydrogenated palm oil (made by Yokozeki Oil & Fat Corp.)

Fifty parts by mass of the hydrogenated palm kernel oil and 50 parts by mass of the fully hydrogenated palm oil as shown in Table 1 were mixed together and completely fused at 70° C. Subsequently, 0.15 parts by mass of “Lipase QLM” (made by Meito Sangyo Co., Ltd.) serving as a lipase enzyme product were added to 100 parts by mass of the oil and fat mixture, to carry out a reaction at 70° C. for 16 hours. The enzyme was removed from the oil and fat mixture by filtration. The oil thus obtained from the reaction was refined by the conventional process, thereby obtaining an oil and fat composition No. 1 having a trans-fatty acid content of 0% by mass, an iodine value of 0.36, and a slip melting point of 44.1° C.

The fatty acid contents and the trans-fatty acid content were determined using a capillary gas chromatography (“HP6890 Gas Chromatography” made by Hewlett-Packard Japan, Ltd.), the temperature being programmed from 100 to 250° C. The iodine value was determined by the Wijs method in accordance with “Standard Methods for the Analysis of Fats, Oils and Related Materials” (by Japan Oil Chemists' Society). The slip melting point was measured in accordance with the method described in “Standard Methods for the Analysis of Fats, Oils and Related Materials” (by Japan Oil Chemists' Society) using a glass capillary with a length of 75 mm (made by Drummond Scientific Company) and an automatic measuring instrument for slip melting point (made by Elex Scientific Co., Ltd.).

### Example 2

Raw materials, that is, an oil and fat (A) and an oil and fat (B) as shown in Table 2 were used to produce an oil and fat composition No. 2.

TABLE 2

| | Oil and Fat (A) | |
|---|---|---|
| | RBD palm kernel | Oil and Fat (B) |
| | oil*¹ | Bleached palm oil*² |
| Content of fatty acids | 69.4% by mass | 2.2% by mass |
| (C14 or less) | | |
| Content of fatty acids | 53.6% by mass | — |
| (C12 or less) | | |
| Content of fatty acids | 30.6% by mass | 97.8% by mass |
| (C16-C24) | | |
| Trans-fatty acid content | 0.1% by mass | 0.3% by mass |
| Iodine value | 17.8 | 52.7 |

*¹Commercially available product name: RBD palm kernel oil (made by Mitsubishi Corporation.)
*²Commercially available product name: Bleached palm oil (made by The Nisshin Oilio Group, Ltd.)

Fifty parts by mass of the RBD palm kernel oil and 50 parts by mass of the bleached palm oil as shown in Table 2 were mixed together and completely fused at 50° C. Subsequently, 0.2 parts by mass of “Lipase QLM” (made by Meito Sangyo Co., Ltd.) serving as a lipase enzyme product were added to 100 parts by mass of the oil and fat mixture, to carry out a reaction at 70° C. for 16 hours. The enzyme was removed from the oil and fat mixture by filtration. The oil thus obtained from the reaction was refined by the conventional process, heated to 120° C. with stirring under reduced pressure to remove the water content. After that, a catalyst for hydrogenation, “SO-850” (made by Sakai Chemical Industry Co., Ltd) in an amount of 0.2 parts by mass was added to 100 parts by mass of the above-mentioned oil, to carry out a hydrogenation reaction at a reaction temperature of 185° C. under a hydrogen pressure of 0.2 MPa with stirring at 300 rpm for one hour. After completion of the reaction, the catalyst was removed by filtration and the hydrogenated (hardened) oil thus obtained was refined by the conventional process, thereby obtaining an oil and fat composition No. 2 having a trans-fatty acid content of 0% by mass, an iodine value of 0.33, and a slip melting point of 44.2° C.

### Comparative Example 1

Fifty parts by mass of the same hydrogenated palm kernel oil (available from The Nisshin Oilio Group, Ltd. under the trade name of Hydrogenated palm kernel oil) as in Example 1 and 50 parts by mass of the same fully hydrogenated palm oil (available from Yokozeki Oil & Fat Corp. under the trade name of Fully hydrogenated palm oil) as in Example 1 were mixed together and completely fused at 70° C., thereby obtaining an oil and fat composition No. 3 having a trans-fatty acid content of 0% by mass, an iodine value of 0.34, and a slip melting point of 46.9° C.

Table 3 shows the solid fat contents for the oil and fat compositions Nos. 1, 2 and 3 respectively obtained in Examples 1 and 2 and Comparative Example 1 at various temperatures within a range from 5 to 50° C. The solid fat contents were measured using a measuring instrument for the solid fat content based on pulsed nuclear magnetic resonance technology (made by Resonance Instruments Ltd.).

TABLE 3

| | Example 1 | | Comparative |
|---|---|---|---|
| Solid Fat | Oil and fat | Example 2 | Example 1 |
| Contents | composition | Oil and fat | Oil and fat |
| (%) | No. 1 | composition No. 2 | composition No. 3 |
| 5° C. | 95.8 | 95.9 | 95.5 |
| 10° C. | 94.9 | 95.2 | 95.0 |
| 15° C. | 94.4 | 94.3 | 94.1 |
| 20° C. | 93.4 | 93.4 | 91.2 |
| 25° C. | 91.4 | 91.4 | 85.4 |
| 30° C. | 83.2 | 82.9 | 77.1 |
| 35° C. | 66.6 | 66.3 | 66.6 |
| 40° C. | 39.8 | 40.0 | 56.7 |
| 45° C. | 17.0 | 16.9 | 46.7 |
| 50° C. | 0.9 | 0.7 | 30.0 |

As can be seen from the results in Table 3, the obtained oil and fat compositions are substantially the same and there is no difference in the final embodiment even though the order of transesterification and hydrogenation is reversed in the course of the preparation of the oil and fat composition.

### Example 3

Twelve parts by mass of the oil and fat composition No. 1 obtained in Example 1 were mixed with 88 parts by mass of a canola oil with high oleic acid content and low linolenic acid content, thereby obtaining a fat for margarine. After that, 84 parts by mass of the fat for margarine and 16 parts by mass of water phase were mixed to form an emulsion. The emulsion was rapidly cooled while being kneaded in a heat transfer apparatus “ONLATOR” (made by Sakura Seisakusho, Ltd.), so that a margarine No. 1 was obtained. The margarine fat had a saturated fatty acid content of 16.4% by mass and a trans-fatty acid content of 0.6% by mass.

### Example 4

The procedure for preparation of the margarine No. 1 in Example 3 was repeated except that the oil and fat composition No. 1 was replaced by the oil and fat composition No. 2, so that a margarine No. 2 was obtained. The margarine fat had a saturated fatty acid content of 16.3% by mass and a trans-fatty acid content of 0.6% by mass.

### Comparative Example 2

The procedure for preparation of the margarine No. 1 in Example 3 was repeated except that the oil and fat composition No. 1 was replaced by the oil and fat composition No. 3, so that a margarine No. 3 was obtained. The margarine fat had a saturated fatty acid content of 16.3% by mass and a trans-fatty acid content of 0.6% by mass.

The margarines Nos. 1, 2 and 3 respectively produced in Examples 3 and 4 and Comparative Example 2 were evaluated in terms of ease of spreading, melting texture and flavor, and cone penetration. The evaluation results are shown in Table 4. The evaluation of the ease of spreading and melting texture and flavor was repeated three times using 15 panelists.

TABLE 4

| | | | Comparative |
|---|---|---|---|
| | Example 3 | Example 4 | Example 2 |
| | Margarine No. 1 | Margarine No. 2 | Margarine No. 3 |
| Ease of | soft and easily | soft and easily | too hard to |
| spreading | spreads. | spreads. | smoothly spread |
| | | | and becomes |
| | | | rolled up. |
| Melting | melts smoothly | pleasantly | partially |
| texture and | and pleasantly. | melts and has | remains in the |
| flavor | | refreshing | mouth as a wax. |
| | | flavor. | unpalatable |
| | | | taste. |
| Cone | 5° C.: 234.6 | 5° C.: 233.7 | 5° C.: 185.5 |
| penetration | 10° C.: 260.8 | 10° C.: 259.0 | 10° C.: 196.6 |

The margarines No. 1 and No. 2 (obtained in Examples 3 and 4 respectively) were both excellent in the spreadability and the melting texture. In contrast to this, the spreadability and the melting texture of the margarine No. 3 (obtained in Comparative Example 2) were found to be unsatisfactory for a food product.

What is claimed is:

1. A method for producing an oil and fat composition comprising the steps of:
(a) mixing an oil and fat (A) wherein fatty acids with 14 or less carbon atoms are contained in an amount of 50 to 100% by mass of entire constituent fatty acids of triglycerides with an oil and fat (B) wherein fatty acids with 16 to 24 carbon atoms are contained in an amount of 80 to 100% by mass of entire constituent fatty acids of triglycerides to obtain an oil and fat mixture;
(b) subjecting the oil and fat mixture to random transesterification using an enzyme having no positional specificity; and
(c) obtaining an oil and fat composition free from trans fatty acids.

2. The method of claim 1, further comprising the step of individually subjecting the oil and fat (A) and the oil and fat (B) to hydrogenation until their iodine value reaches 5 or less, prior to the step (a).

3. The method of claim 1, further comprising the step of subjecting the oil and fat (A) to hydrogenation until its iodine value reaches 5 or less, prior to the step (a).

4. The method of claim 1, further comprising the step of subjecting the oil and fat (B) to hydrogenation until its iodine value reaches 5 or less, prior to the step (a).

5. The method of claim 1, further comprising the step of subjecting the oil and fat mixture to hydrogenation, prior to the step (b).

6. The method of claim 1, further comprising the step of carrying out hydrogenation after the step (b).

7. The method of claim 1, wherein the oil and fat (A) is an oil and fat wherein fatty acids with 12 or less carbon atoms are contained in an amount of 45 to 100% by mass of the entire constituent fatty acids of the triglycerides.

8. The method of claim 1, wherein a ratio by mass of the oil and fat (A) to the oil and fat (B) is in the range of 4:6 to 8:2.

9. The method of claim 1, wherein a ratio by mass of the oil and fat (A) to the oil and fat (B) is in the range of 4:6 to 6:4.

10. The method of claim 1, wherein the oil and fat (A) is selected from the group consisting of coconut oil, palm kernel oil, fractionated products thereof, medium chain triglycerides, and mixtures thereof.

11. The method of claim 1, wherein the oil and fat (B) is selected from the group consisting of soybean oil, rapeseed oil, cottonseed oil, palm oil, palm fractions, and mixtures thereof.

12. The method of claim 1, wherein the enzyme having no positional specificity is lipase.

13. The method of claim 12, wherein the lipase is not immobilized.

14. The method of claim 1, wherein the trans fatty acids in the resulting oil and fat composition is present in an amount of 0% by mass.

15. An oil and fat composition obtainable by a method of claim 1.

16. A method for producing a processed oil and fat product, comprising using the oil and fat composition obtainable by a method of claim 1.

17. A processed oil and fat product comprising the oil and fat composition obtainable by a method of claim 1.

18. The processed oil and fat product of claim 17, wherein the contents of the saturated fatty acids in the fatty acids constituting entire oil and fats contained in the processed oil and fat product is in the range of 8 to 60% by mass.

19. The processed oil and fat product of claim 17, wherein the processed oil and fat product is a margarine or shortening.
